(19) United States
(12) Patent Application Publication
(10) Pub. No.: US 2026/0124024 A1
(43) Pub. Date: May 7, 2026

(54) DENTAL AND SUBPERIOSTEAL IMPLANTS COMPRISING BIOCOMPATIBLE GRAFT

(71) Applicant: CORNEAT VISION LTD, Raanana (IL)

(72) Inventors: GILAD LITVIN, Moshav Sde Varburg (IL); ALMOG ALEY-RAZ, Moshav Sde Varburg (IL); PATRICIA ASSOULINE, Kafar Yona (IL)

(73) Assignee: CORNEAT VISION LTD, Raanana (IL)

(21) Appl. No.: 19/117,431

(22) PCT Filed: Oct. 2, 2023
(86) PCT No.: PCT/IL2023/051054

§ 371 Date: Apr. 1, 2025

Related U.S. Application Data
(60) Provisional application No. 63/412,567, filed on Oct. 3, 2022.

(51) Int. Cl. A61C 8/02; A61C 8/00; A61K 6/69; A61K 6/891

(52) U.S. Cl. CPC A61C 8/0006; A61C 8/0016; A61C 8/0037; A61K 6/69; A61K 6/891; A61C 2008/0046; A61C 2201/00

(57) ABSTRACT

The invention provides a dental or subperiosteal implant having a porous polymeric structure and uses thereof in the method of treatment of periodontal diseases, conditions and symptoms.

### BACKGROUND OF THE INVENTION

Rehabilitation of edentulous patients either with or without atrophic jaws is a surgical challenge. Conventional endo-osseous implant-supported overdentures and immediate loading protocols still presents a clinical challenge nowadays. Many techniques have been described in the literature to overcome this problem. Reconstructive procedures, such as autologous bone grafting or guided bone regeneration, are often used. However, autogenous bone grafting requires a second surgical site, implying additional morbidity, and immediate loading is not always recommended. Guided bone regeneration, particularly vertical, is frequently limited in gain and also associated with possible complications in total atrophic jaws. Both techniques require several months for graft maturation. Following implantation, dental implants can present with peri-implnatitis, years following their implantation, necessitating surgical intervention and occasionally removal of the implant. Atrophic jaws are associated with anatomical changes, carrying an increased risk of injury to noble structures, thus increasing the needs of specific surgical skills during surgery.

There is a need to provide a safe and reliable dental implant or dental subperiosteal implant that can provide a permanent solution for edentulous and atrophic jaws and related diseases and conditions without the risk of inflammation, implant exposure and extrusion, peri-implantitis, infection and eventually loss of the implant and the need for further surgeries.

### SUMMARY OF THE INVENTION

The present invention provides an effective and stable solution for implanting dental implants and subperiosteal implants that are on the one hand versatile, making the procedure simple to perform, and on the other hand bio-stable and biocompatible, having an improved and lasting effect and providing effective integration with the jaw and dental tissue.

The invention provides a dental or subperiosteal implant comprising at least one synthetic biocompatible graft having a porous polymeric structure with pores of less than 5 microns.

When referring to “subperiosteal implant” it should be understood to refer to a metal implanted framework that rests directly on top of the bone, underlying the periosteum, and providing attachment posts, which extend through the gingival tissue for prosthesis anchorage.

When referring to “dental implants” it should be understood to refer to a prosthesis that interfaces with the bone of the jaw or skull to support a dental prosthesis such as a crown, bridge, denture, or facial prosthesis or to act as an orthodontic anchor. Dental implants are typically formed from materials such as titanium or zirconia and form an intimate bond to the bone they are implanted in. The implant fixture is first placed so that it is likely to osseointegrate, then a dental prosthetic is added. A variable amount of healing time is required for osseointegration before either the dental prosthetic (a tooth, bridge, or denture) is attached to the implant or an abutment is placed which will hold a dental prosthetic/crown. The prerequisites for long-term success of osseointegrated dental implants are healthy bone and gingiva. Since both can atrophy after tooth extraction, pre-prosthetic procedures such as sinus lifts or gingival grafts are sometimes required to recreate ideal bone and gingiva.

A typical conventional implant (shown in FIG. 1) comprises a root implanted part (103) made of metal (usually made of titanium or a titanium alloy) in the form of a screw (resembling a tooth root) with a roughened or smooth surface and a top part (101) resembling a tooth (or teeth) made for example from zirconia. Abutment (102) connects between the implanted root part and the upper visible tooth part. The majority of the root implanted part of said dental implants are made of commercially pure titanium, or titanium alloys. Most modern dental implants also have a textured surface (through etching, anodic oxidation or various-media blasting) to increase the surface area and osseointegration potential of the implant. In some embodiments of an implant of the invention shown in FIG. 2, the root implanted part is covered with said synthetic biocompatible graft (106), wherein the abutment (105) and the external tooth part (104) remain uncoated.

Thus, an implant of the invention provides for an advantageous osseointegration.

When referring to a “synthetic biocompatible graft” it should be understood to relate to an implantable being a synthetic polymeric material which is biocompatible with the dental tissue, such as the gingival tissue, wherein cell growth in the vicinity of the graft is capable of growing and integration at the jawbone-gingiva border within said graft. In some embodiments, said graft of the invention is in the form of a sheet.

In some embodiments, said at least one synthetic biocompatible graft covers, envelopes, and/or coats substantially all of the metal framework (metal root implanted part) of said subperiosteal implant or any dental implant. In other embodiments, said at least one synthetic biocompatible graft covers, envelopes and/or coats at least a part of the metal framework of said subperiosteal implant and any dental implant.

In some embodiments, said at least one synthetic biocompatible graft coats at least a part of said implant. In such embodiments, at least one synthetic biocompatible graft coats at least the root implanted part of a dental implant. In other embodiments, at least one synthetic biocompatible graft coats at least the metal implanted framework of a subperiosteal implant of the invention.

In some embodiments, said synthetic biocompatible graft having a porous polymeric structure, has pores of between about 0.01 microns to 5 microns. In some embodiments a synthetic biocompatible graft having a porous polymeric structure, has pores of 0.01, 0.05, 0.1, 0.5, 1, 1.5, 2, 2.5, 3, 3.5, 4, 4.5, 5 microns.

The invention further provides a synthetic biocompatible graft having a porous polymeric structure with pores of between 5 to 20 microns. In some embodiments, a synthetic biocompatible graft having a porous polymeric structure with pores of 5, 6, 7, 8, 9, 10, 11, 12, 13, 14, 15, 16, 17, 18, 19, 20 microns.

In other embodiments, said synthetic biocompatible graft of the invention is a non-degradable graft. In other embodiments, said synthetic biocompatible graft of the invention is a permanent integral non-degradable graft.

In some embodiments, said synthetic biocompatible graft of the invention has a thickness of between 100-1000 μm. In other embodiments, said synthetic biocompatible graft of the invention has a thickness of between 10-100 μm. In other embodiments, said synthetic biocompatible graft of the invention has a thickness of between 1000-2500 μm.

In some embodiments, said porous polymeric structure comprises at least one polymer. In other embodiments, said porous polymeric structure comprises nanofibers (in some embodiments, said nanofibers are 500 nm to a few micrometers in thickness).

In some embodiments, said porous polymeric structure comprises at least one porous electrospun polymer. In some other embodiments said porous polymeric structure comprises at least one porous printed polymer (using for example a 3D printing device).

In some further embodiments, said porous polymeric structure comprises at least one polymer selected from polycarbonate, poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane (including aromatic polyurethane), polyvinyl chloride (PVC), hyaluronic acid (HA), chitosan, alginate, polyhydroxybuyrate and its copolymers, Nylon 11, Cellulose acetate, hydroxyapatite, poly(3-hydroxybutyric acid-co-3-hydroxyvaleric acid), poly(DL-lactide), polycaprolactone, and poly(L-lactide) or any combination thereof.

Electrospun fibers are typically several orders in magnitude smaller than those produced using conventional spinning techniques. By optimizing parameters such as: i) the intrinsic properties of the solution including the polarity and surface tension of the solvent, the molecular weight and conformation of the polymer chain, and the viscosity, elasticity, and electrical conductivity of the solution; and ii) the operational conditions such as the strength of electric field, the distance between spinneret and collector, and the feeding rate of the solution, electrospinning is capable of generating fibers as thin as tens of nanometers in diameter. Additional parameters that affect the properties of electrospun fiber include the molecular weight, molecular-weight distribution and structure (branched, linear etc.) of the polymer, solution properties (viscosity, conductivity and surface tension), electric potential, flow rate and concentration, distance between the capillary and collection screen, ambient parameters (temperature, humidity and air velocity in the chamber), motion of target screen (collector) and so forth. Fabrication of highly porous fibers may be achieved by electrospinning the jet directly into a cryogenic liquid. Well-defined pores developed on the surface of each fiber as a result of temperature-induced phase separation between the polymer and the solvent and the evaporation of solvent under a freeze-drying condition.

Several approaches have been developed to organize electrospun fibers into aligned arrays. For example, electrospun fibers can be aligned into a uniaxial array by replacing the single-piece collector with a pair of conductive substrates separated by a void gap. In this case, the nanofibers tend to be stretched across the gap oriented perpendicular to the edges of the electrodes. It was also shown that the paired electrodes could be patterned on an insulating substrate such as quartz or poly styrene so the uniaxially aligned fibers could be stacked layer-by-layer into a 3D lattice. By controlling the electrode pattern and/or the sequence for applying high voltage, it is also possible to generate more complex architectures consisting of well-aligned nanofibers.

Electrospun nanofibers could also be directly deposited on various objects to obtain nanofiber-based constructs with well-defined and controllable shapes. In addition, one can manually process membranes of aligned or randomly oriented nanofibers into various types of constructs after electrospinning: for example, fabrication of a tube by rolling up a fibrous membrane or the preparation of discs with controllable diameters by punching a fibrous membrane.

The present invention relates to any electrospinning technique known in the art, which includes Electrospinning, J. Stanger, N. Tucker, and M. Staiger, I-Smithers Rapra publishing (UK), An Introduction to Electrospinning and Nanofibers, S. Ramakrishna, K. Fujihara, W-E Teo, World Scientific Publishing Co. Pte Ltd (Jun 2005), Electrospinning of micro-and nanofibers: fundamentals and applications in separation and filtration processes, Y. Fillatov, A. Budyka, and V. Kirichenko (Trans. D. Letterman), Begell House Inc., New York, USA, 2007, which are all incorporated herein by reference in their entirety.

Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference. It is to be understood that although the according to the presently preferred embodiment of the invention is described with a particular emphasis to the electrospinning technique, it is not intended to limit the scope of the invention to the electrospinning technique. Representative examples of other spinning techniques suitable for the present embodiments include, without limitation, a wet spinning technique, a dry spinning technique, a gel spinning technique, a dispersion spinning technique, a reaction spinning technique or a tack spinning technique. Such and other spinning techniques are known in the art and disclosed, e.g., in U.S. Pat. Nos., 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

In order to increase the osteointegration of an implant of the invention, said at least one synthetic biocompatible graft coating said implant has an external surface (that is in contact with bone or tissue at the implanting site) that is rough particulate (uneven) having particle formation on its surface (for example coral like surface). In some embodiments, said at least one synthetic biocompatible graft has a surface that is a rough particulate surface. In some embodiments, said at least one synthetic biocompatible graft comprise electrospun fibers having a coral like rough surface. In some embodiments, the external surface of said at least one synthetic biocompatible graft has a particulated surface having particle size of at least 50 μm. In some embodiments, the surface of said at least one synthetic biocompatible graft has a particle size of at most 50 μm. In some embodiments, the surface of said at least one synthetic biocompatible graft has a particle size of 1 to 50 μm. In some embodiments, said at least one synthetic biocompatible graft comprise electrospun fibers that have been post-treated with cryogenic grinding.

In some embodiments, said synthetic biocompatible graft of the invention further comprises at least one active agent. In some embodiments, said at least one active agent is selected from a protein, type I collagen, fibronectin, or TGF-beta 2, heparin, growth factors, antibodies, antimetabolites, chemotherapeutic agents, anti-inflammatory agent, anti-biotic agent, and any combinations thereof.

In some embodiments, said synthetic biocompatible graft of the invention is cut into a designated shape (in some embodiments, said cut is laser cut, manual cut, pressure cut and so forth).

In some embodiments, said synthetic biocompatible graft of the invention further comprises at least one non-porous layer. In some embodiments, said at least one non-porous layer is in the form of a film. When used as a tissue replacement in periodontal surgery, said at least one non-porous layer or film is placed on the bone side of the cavity to be filled.

When referring to a “non-porous layer” it should be understood to encompass a film layer that has substantially no pores, thus not penetrable by tissue, impervious as compared with said porous layer of the biocompatible graft of the invention.

In some embodiments, said non-porous layer is a biocompatible graft. In other embodiments, said non-porous layer is a non-degradable graft.

In some embodiments, said non-porous layer has a thickness of between 100-1000 μm. In other embodiments, said non-porous layer has a thickness of between 10-100 μm. In other embodiments, said non-porous layer has a thickness of between 1000-2500 μm.

In some embodiments, said non-porous polymeric structure comprises at least one polymer. In other embodiments, said non-porous polymeric structure comprises nanofibers (in some embodiments, said nanofibers are 500 nm to a few micrometers in thickness).

In some further embodiments, said non-porous polymeric structure comprises at least one polymer selected from polycarbonate, poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane (including aromatic polyurethane), polyvinyl chloride (PVC), hyaluronic acid (HA), chitosan, alginate, polyhydroxybuyrate and its copolymers, Nylon 11, Cellulose acetate, hydroxyapatite, poly(3-hydroxybutyric acid-co-3-hydroxyvaleric acid), poly(DL-lactide), polycaprolactone, and poly(L-lactide) or any combination thereof.

When relating to “periodontal disease, condition or symptom” it should be understood to encompass inflammatory conditions affecting the tissues surrounding the teeth such as for example gum disease, gingivitis, periodontitis, tooth decay, tooth loss, bone loss, peri-implnatitis and any combinations thereof.

In some embodiments, such periodontal disease, condition or symptom may cause the need for periodontal surgery.

The term “periodontal surgery” is meant to encompass a form of dental surgery that prevents, corrects or reconstructs anatomical, traumatic, developmental, age related or plaque-induced defects in the bone, gingiva, or alveolar mucosa, maxillofacial surgery and any combinations thereof. The objectives of this surgery include accessibility of instruments to root surface, elimination of inflammation, creation of an oral environment for plaque control, periodontal diseases control, oral hygiene maintenance, maintain proper embrasure space, address gingiva-alveolar mucosa problems, and esthetic improvement. The surgical procedures include among others, crown lengthening, frenectomy, mucogingival flap surgery, gingivectomy, apically repositioned flap (APF) surgery, apically repositioned flap (APF) with osseous reduction (osteoplasty/ostectomy) and any combinations thereof.

The invention further provides a synthetic biocompatible graft as disclosed herein above and below for use in periodontal and/or dental surgery.

The invention further provides a synthetic biocompatible graft as disclosed herein above and below for use in the treatment of periodontal injury, disease, condition or symptom.

The invention further provides a device comprising a dental or subperiosteal implant of the invention.

The invention further provides a kit comprising a dental or subperiosteal implant of the invention, means for its periodontal implanting/placement in the gingival tissue of a subject and instructions for use.

In some embodiments, instructions of use may include: instructions for the care giver how to custom cut the graft of the invention, how to place said graft invention for example, over the teeth and on the gingival tissue.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows a typical dental crown implant.

FIG. 2 shows a typical dental crown implant covered with a biocompatible graft.

FIGS. 3A-3B show surgical sites at termination (3-weeks post-op) of Example 1.

FIGS. 4A-4C show the histological Slide (H&E) of a non-treated site at 6 weeks post-op (B—bone tissue; CT—connective tissue; Ep—Epithelium) of Example 2.

FIGS. 5A-5D show the histological Slide (H&E) of a patch of the invention implanted site at 3 weeks post-op (Asterisk-marks the patch) of Example 3.

FIGS. 6A-6F show the histological slides (H&E) of surgery site at 10 weeks post-op (Asterisk—Synthetic biocompatible graft) of Example 4.

FIG. 7 shows the test models of Example 5.

FIGS. 8A-8D show the histological slides—Sinclair MiniPigs, patch of the invention implanted sites, 1 month post implantation.

FIGS. 9A-9B demonstrate SEM images of the particles obtained via post processing of electrospun fibers to coral like rough surface.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

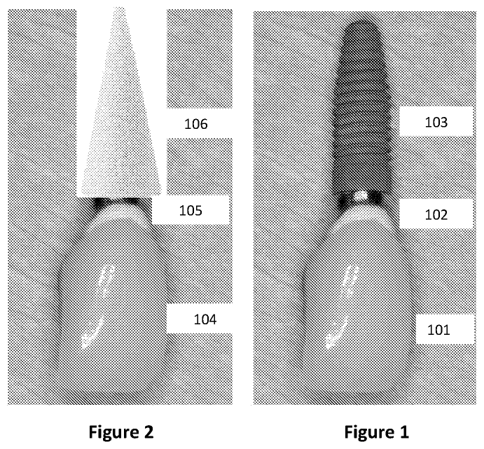

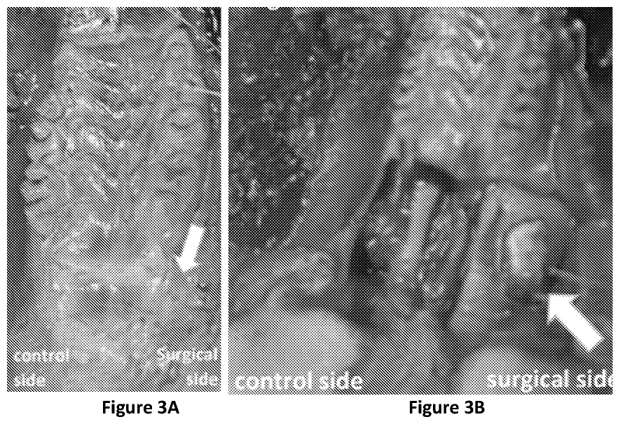

FIG. 1 shows a typical dental plant having the external crown and the metal implant that is inserted into the bone of the jaw. FIG. 2 shows the crown dental implant wherein the metal implant that is inserted into the bone of the jaw is covered/enveloped/coated by a biocompatible graft.

### Example 1—Evaluate Oral Implantation of the Implant of the Invention in a Rat Model, Assessing Its Integration with Gingival and Bone Tissues

Experimental Model: A single rat was chosen as the animal model to assess the feasibility of the synthetic biocompatible graft's implantation in an oral environment and its performance. The synthetic biocompatible graft, sized at 2×2 mm, was implanted into the gums of the rat's upper jaw on the right side. Over the subsequent three-week observation period, the rat exhibited normal eating habits, indicating a lack of discomfort or impairment.

Results: Upon examination at the end of the study, the gingival tissue at the implantation site showed signs of complete healing, resembling the non-treated area.

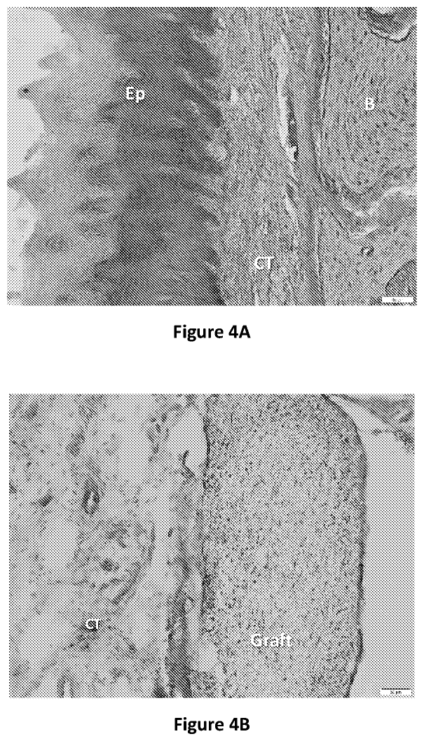

Notably, the synthetic biocompatible graft was visibly present beneath the gingival tissue, indicating successful implantation. However, during tissue dissection, it was observed that the synthetic biocompatible graft was more integrated with the adjacent bone tissue than with the soft tissue. Although it wasn't fully integrated into the gingival tissue, removing it was difficult, suggesting strong integration with the bone. FIGS. 3A and 3B show the surgical sites at termination (3-weeks post-op). FIG. 3A shows the upper jaw (surgical site) prior to dissection is depicted, showing no macroscopical adverse events. This is evident from the similarity in tissue appearance compared to the non-operated contralateral side. FIG. 3B shows the surgical site immediately after tissue dissection. The Synthetic biocompatible graft is clearly visible, indicated by the arrow, and appears to be securely attached to the bone beneath the gingiva.

Conclusion: The Synthetic biocompatible graft demonstrated excellent wound healing and integration with bone tissue. However, integration with soft tissue was comparatively lower.

### Example 2—Evaluation of the Synthetic Biocompatible graft's Effectiveness in a Gingival Recession Model and Assess the Acute Healing of Both Soft and Bony Tissues

Experimental Model: The study involved six SD rats, equally distributed for termination at 3 and 6 weeks post-operation. Each rat underwent an implantation procedure of the Test Device within a gingival pouch formed on the right side of the maxilla. This involved creating a pocket 3 mm wide and 3 mm deep using a 15c blade anteriorly to the upper right molars. Subsequently, a patch measuring 2×2 mm was placed into the buccal aspect of the formed pocket, followed by defect closure using Vicryl 5-0 sutures.

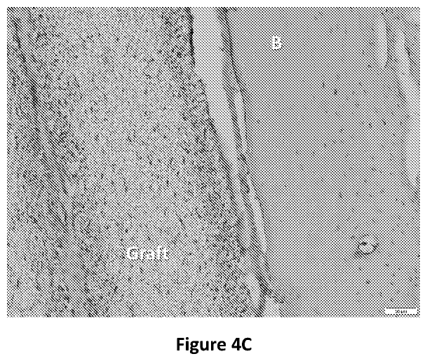

Results: At both the 3-week and 6-week checkpoints, the animals displayed signs of successful healing, with the exception of one rat that experienced patch exposure. Histological analysis provided significant evidence of cell infiltration from adjacent bone tissue, indicating a robust integration between the implanted synthetic biocompatible graft and the bony tissue. This suggests that the synthetic biocompatible graft shows promise for integration into bone tissue and may be a viable option for similar applications. FIGS. 4A-4C show the histological Slide (H&E) of a non-treated site at 6 weeks post-op (B-bone tissue; CT-connective tissue; Ep-Epithelium). FIG. 4B shows that the patch is clearly identified, interfacing with soft tissue. Remarkably, there is a notable absence of inflammatory cell reaction, indicating biocompatibility. FIG. 4C shows that the patch is in contact with bony tissue. Similar to FIG. 4B, there is a lack of inflammatory cell reaction. Moreover, the figure underscores cell infiltration into the porous matrix from both soft and bony tissue, with a discernible increase in cell infiltration at the interface with bony tissue, suggesting more robust cellular integration in this region.

Conclusion: In conclusion, the histological analysis yielded compelling evidence of substantial cell infiltration originating from neighboring bone tissue, signifying a strong integration between the implanted patch and the bony substrate. This finding supports the potential suitability of the Synthetic biocompatible graft for applications involving integration into bone tissue.

### Example 3—Rat Calvarial Defect—Model validation

Experimental Model: Three rats were involved, each undergoing sub-periosteal implantation of an oversized rectangular Synthetic biocompatible graft over a 5 mm bone defect in the calvarial bone. Animals were observed for a duration of 3 weeks after implantation.

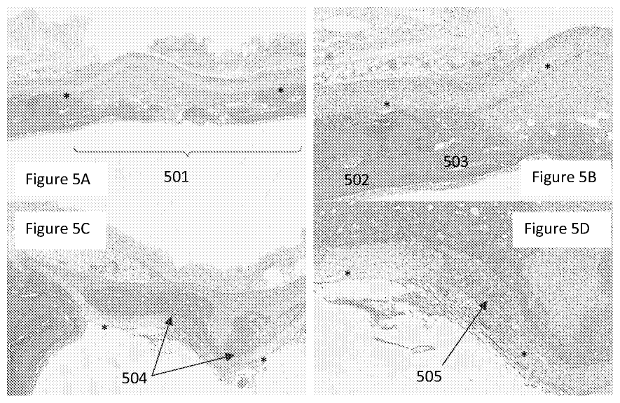

Results: Over the course of three weeks, there were no observed adverse events, and all animals exhibited normal weight gain. Macroscopic examination at termination revealed that two out of the three patches remained in good position and location, while one patch appeared to be partially dislocated towards the nasal area. Histological evaluation indicated moderate cell infiltration into the patch, primarily at the aspect facing the bony tissue. Osteogenesis at the defect site was noted in all animals, with significant osteogenesis noted over the patch in the animal where the graft was misplaced. FIGS. 5A-5D show the histological Slide (H&E) of a patch of the invention implanted site at 3 weeks post-op (Asterisk-marks the patch). FIGS. 5A and 5B are extracted from a histological slide of animal #1's implantation site provide a view at both low (5A) and high (5B) magnifications. The patch is distinctly observable, showing no signs of inflammatory reaction. 501 marks the bone defect. 502 marks the old bone and 503 marks the new bone formed. Notably, there is observable new bone formation at the periphery of the bone defect, and a notable infiltration of cells into the porous matrix. FIGS. 5C and 5D are derived from a histological slide of animal #2's implantation site, where the implant appeared to be partially displaced. It was observed that both low (5C) and high (5D) magnifications. In this case, osteogenesis is evident above the patch and extending into the patch itself indicating the potential of the patch to promote bone formation. 504 marks the new bone formation over the patch and 505 marks the new bone formation inside the patch.

Conclusion: The synthetic biocompatible graft demonstrated overall safety and the potential to induce bone regeneration.

### Example 4—Assessment of Bone Regeneration using the Rat Calvarial Defect Model

Experimental Model: This study involved a larger cohort of 10 rats, which were divided into three test groups as follows:
Group #1—received Synthetic biocompatible graft implantation directly onto the calvarial bone.
Group #2—received Synthetic biocompatible graft implantation onto a calvarial defect.
Group #3—served as a control with a bone defect but no Synthetic biocompatible graft implantation.

Animals were observed for 10 weeks after implantation.

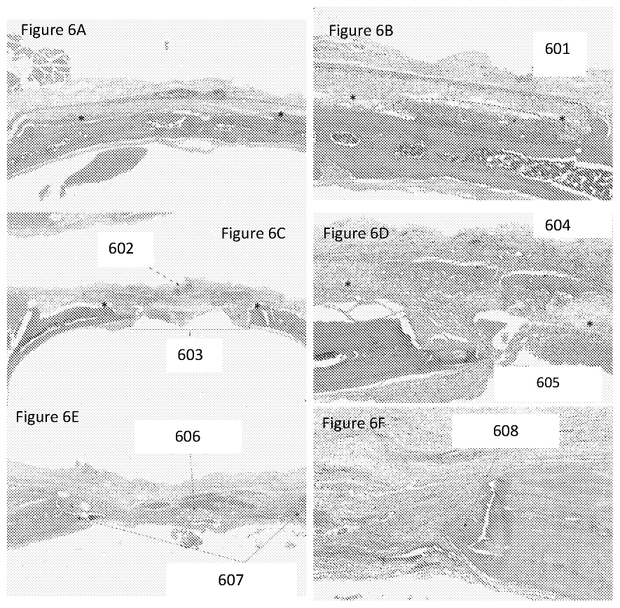

Results: Over a 10-week follow-up period, no systemic or local reactions were noted. Macroscopic evaluation upon termination showed that all patches remained in place. Histological analysis revealed significant cell growth into the patch's pores and even mild osteogenesis into the patch. Group #2 demonstrated new bone formation above the patch, indicating that the synthetic biocompatible graft has the potential to promote bone regeneration compared to untreated bone defects. FIGS. 6A-6F show the histological slides (H&E) of surgery site at 10 weeks post-op (Asterisk-Synthetic biocompatible graft).

FIGS. 6A and 6B were extracted from a histological slide of animal #1 in Group #1, providing both low (6A) and high (6B) magnifications. The synthetic biocompatible graft is prominently visible with no signs of inflammatory reaction. Remarkably, new bone formation is observed growing into the synthetic biocompatible graft. Additionally, note the tares in the patch, which are an artifact of histological processing, indicating a robust connection to the underlying bone. 601 marks the new bone formed. FIGS. 6C and 6D were obtained from a histological slide of animal #2 in Group #2, offering both low (6C) and high (6D) magnifications. Notably, significant bone regeneration is apparent, originating from the edge of the bone defect and extending through the synthetic biocompatible graft, even above it. At the defect's edge, there is observable tissue discoloration, potentially indicative of thermal osteonecrosis, likely a result of drill overheating. 602 marks the sutures, 603 marks the bone defect. 604 marks the new bone growing through and over the patch and 605 marks the suspected thermal osteonecrosis, probably due to overheating of the drill.

FIGS. 6E and 6F were derived from a histological slide of animal #1 in Group #3, presenting both low (6E) and high (6F) magnifications. In this case, minimal osteogenesis is noted, while soft tissue fills the bone defect. 606 marks the soft tissue fills that fills the gap with no new bone formation. 607 marks the suspected thermal osteonecrosis (due to the drill). 608 marks the minimal new bone formation.

Conclusion: This study suggests that the synthetic biocompatible graft holds promise for inducing bone regeneration and may offer advantages over untreated defects.

### Example 5—Mandibular Defect Model in Miniature Swine

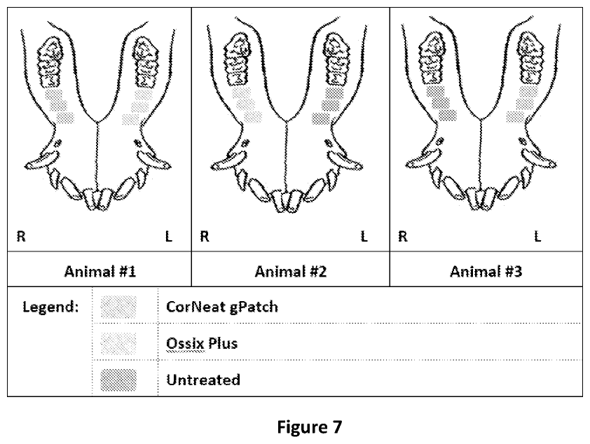

Experimental Model: This study involved a more complex animal model, utilizing miniature swine to evaluate the therapeutic effect of the synthetic biocompatible graft on mandibular defects. The study incorporated in-life and post-mortem assessments to assess osteointegration and new bone growth. The study consisted of 3 Sinclair mini pigs, initially subjected to an extraction of six mandibular pre-molar teeth. After a healing period of 10 weeks, three (3), ˜7×8×10 mm3 alveolar defects per hemi-mandible were created, for a total of 6 defects per animal. Following their creation, the defects were filled with a commercial bone filler (Bio-Oss, Geistlich) and covered with either the Test (CorNeat gPatch of the invention) or Reference Item (Ossix Plus) or left untreated per the scheme in FIG. 7.

Results: Abnormalities related to the synthetic biocompatible graft were observed in both animals, including exposure of the membrane and possible infection, leading to difficulties in eating and weight loss. Post-mortem findings supported the clinical observations, indicating enlarged and reactive mandibular lymph nodes in the Synthetic biocompatible graft-implanted hemi-mandibles. Ultimately, the study was prematurely terminated due to safety concerns, and the Synthetic biocompatible graft's safety was not demonstrated under these specific conditions. The observed adverse events in the study may be attributed to a combination of factors. Firstly, the thickness (250 microns) and shape memory of the synthetic biocompatible graft could have led to nonconformance with the mandibular implantation surface, potentially hindering integration with surrounding tissues and resulting in implant exposure. Additionally, the surgical technique employed during implantation may have influenced outcomes, as certain techniques, particularly with non-degradable membranes, have been associated with an increased risk of wound edge separation (dehiscence) and subsequent implant exposure. Adopting improved surgical techniques could enhance implant stability and integration, thereby reducing the likelihood of adverse events. Lastly, the choice of minipigs as the animal model might have contributed to the observed outcomes. Differences between minipigs and the more commonly used canine model could impact the device's performance in the oral environment, particularly in terms of daily oral hygiene practices, which were not feasible in this animal model and may have influenced the implant's response and integration with oral tissues.

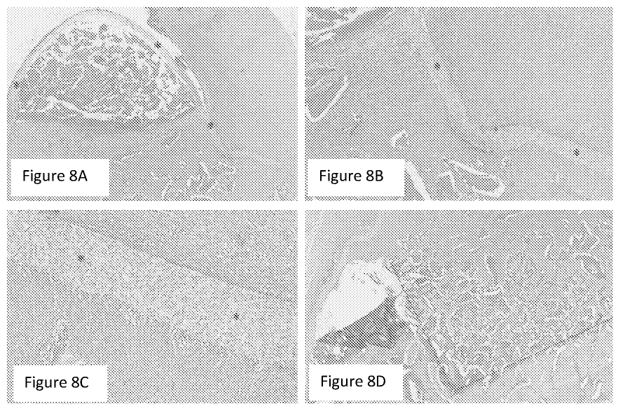

Histological processing and evaluation were performed on the harvested jaw samples. Despite the macroscopic dehiscence of the soft tissue observed during examination, the histological analysis revealed that the synthetic biocompatible graft was strongly fixated to the inner soft tissue and bony tissue. This finding indicates the potential of the Synthetic biocompatible graft to serve as a good solution for Guided Bone Regeneration (GBR) and Guided Tissue Regeneration (GTR) indications. FIGS. 8A-8D show the histological slides—Sinclair MiniPigs, patch of the invention implanted sites, 1 month post implantation.

FIGS. 8A and 8B show H&E low and medium magnification, respectively. Cross-section of the porous polymer (blue asterisks) FIG. 8C shows the H&E in very high magnification demonstrating the patch of the invention (blue asterisks). Note the presence of fibroblasts within the porous polymer indicative of tissue integration. FIG. 8D shows the H&E high magnification demonstrating the borders of the bone defect (dashed red line) and the osteogenesis process within it.

Conclusion: While adverse events were observed during the observation period of the study, the positive histological findings present a promising outlook for the potential efficacy of the Synthetic biocompatible graft in Guided Bone Regeneration (GBR) and Guided Tissue Regeneration (GTR) applications. These results further underscore the importance of its continued development as a valuable solution in dental and regenerative medicine, holding great promise for improving patient outcomes in procedures requiring tissue regeneration and enhancement.

### Example 6—Post Processing of Electrospun Fibers to Coral Like Rough Surface

Cellular attachment to different surfaces is affected by multiple characteristics of the surface such as surface topography (roughness versus smooth), electrical charge, chemistry, porosity and etc. when examining orthopedic implants and dental implants made of titanium or other metallic alloys there is a need to improve the adhesion of bone or connective tissue to such implants to make them more resistant to peri-implantitis or other failure factors associated with such implants. Many methods are known to improve / change the surface characteristics of titanium implants either physically or chemically, however such methods still are not shown to provide a full proof solution for successful implant retention. Biomaterial coatings of implants can increase the implant integration with neighboring tissue. Such biomaterial can either be mineral based to increase bone growth, antibacterial to inhibit bacterial colonization or other.

The electrospun fibers with post processing treatment is used to provide a coral like structure which provides increased surface roughness while maintaining fiber like architecture and mechanical support. Furthermore, the synthetic nature of the biomaterial allows it to further be processed and impregnated with different functional molecules by demand. The biomaterial can easily be incorporated (terminally) into the implant surface and crosslinked enabling long term functional coating of the implant.

Fibroblasts have been documented in the past to prefer adhering to smooth surfaces due to the increased focal adhesion points of such surfaces. However, such smooth surfaces on implants have been shown to promote scar tissue formation (differentiation of fibroblasts into myofibroblasts type cells). Furthermore, roughened surfaces provide higher surface to volume ratio and together with other functionalities of the material can balance the reduced adhesion pattern of fibroblasts on such surfaces. Osteoblasts were previously documented to prefer binding rough surfaces, and this surface topography has been shown to be beneficial in promoting bone formation. Indeed, coral material has been used and is used as a bone formation matrix material.

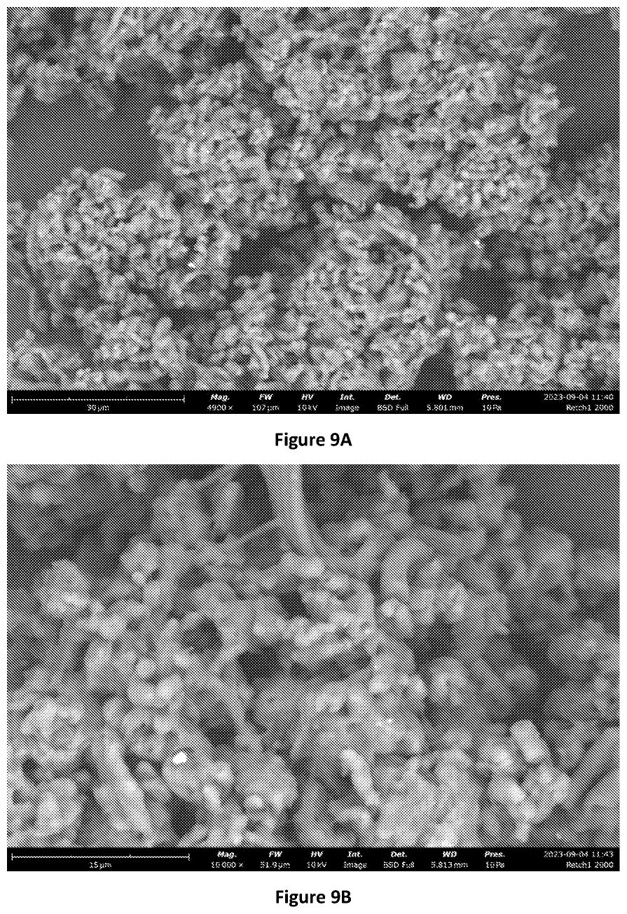

It was demonstrated that the applicability of Cryogenic (liquid nitrogen) grinding of polyurethane electrospun mesh to provide ˜50 μm size particles with rough surface architecture. The raw material was first cooled using liquid nitrogen. The details of the process can be found in the attached report by RETCH. The process demonstrates that a polyurethane mesh can be reduced to 50 μm average particle size. The particles can further be reduced in size by different methods of screening to obtain lower size particles. In addition, the raw material itself can be changed (i.e., thickness) to possibly obtain different particle size dispersion. FIGS. 9A-9B demonstrate SEM images of the particles obtained via this method. FIGS. 9A and 9B show the low (9A) and high (9B) magnification of electrospun fibers post grinding showing roughness of the base material post grinding with fiber architecture semi-intact. The particle raw material is a promising candidate raw material to be tested as coating material. High porosity is achieved based on particle to particle free space as well as inter-particle native pores.

Principle of coating dental implants using polyurethane particles: The Particles are assumed to be activated via plasma treatment to enhance the hydrophilicity of the particles via addition of carboxylic or hydroxy groups (COOH/OH). The metallic implant can be processed in a similar fashion or via alkali acidic treatment. The particles are soluble in aqueous media and will adhere to the implant surface via van der Waals interaction or hydrogen bonds. The particles should be further crosslinked, and one possible crosslinking mechanism can be solvent based gas crosslinking (exposure of the coated implant to solvent vapors for a time dependent crosslinking) this crosslinking is done in room temperature and presents numerous advantages over other heat based or chemical crosslinking modalities. It is important to understand other functional groups can be added by demand to the particle material or implant material to facilitate other attachment mechanisms such as silane based covalent crosslinking or other. This process provides high flexibility in the manufacturing process.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A dental or subperiosteal implant comprising at least one synthetic biocompatible graft having a porous polymeric structure with pores of less than 5 microns.

2. A dental or subperiosteal implant according to claim 1, wherein said pores are between about 0.01 microns to about 5 microns.

3. (canceled)

4. A dental or subperiosteal implant according to claim 1, wherein said synthetic biocompatible graft is a biocompatible graft.

5. A dental or subperiosteal implant according to claim 1, wherein said synthetic biocompatible graft is a non-degradable graft.

6. A dental or subperiosteal implant according to claim 1, wherein said at least one synthetic biocompatible graft coats at least a part of said implant.

7. A dental or subperiosteal implant according to claim 1, wherein said synthetic biocompatible graft has a thickness of between 10-100 μm.

8. A dental or subperiosteal implant according to claim 1, wherein said synthetic biocompatible graft has a thickness of between 100-1000 μm.

9. A dental or subperiosteal implant according to claim 1, wherein said synthetic biocompatible graft has a thickness of between 1000-2500 μm.

10. A dental or subperiosteal implant according to claim 1, wherein said porous polymeric structure comprises at least one polymer.

11. A dental or subperiosteal implant according to claim 1, wherein said porous polymeric structure comprises nanofibers.

12. A dental or subperiosteal implant according to claim 1 wherein said porous polymeric structure comprises at least one porous electrospun polymer.

13. A dental or subperiosteal implant according to claim 1, wherein said porous polymeric structure comprises at least one polymer selected from polycarbonate, poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane, polyvinyl chloride (PVC), hyaluronic acid (HA), chitosan, alginate, polyhydroxybuyrate and its copolymers, Nylon 11, Cellulose acetate, hydroxyapatite, poly(3-hydroxybutyric acid-co-3-hydroxyvaleric acid), poly(DL-lactide), polycaprolactone, and poly(L-lactide) or any combination thereof.

14. A dental or subperiosteal implant according to claim 1, wherein said synthetic biocompatible graft further comprises at least one active agent.

15. The dental or subperiosteal implant according to claim 1, wherein said synthetic biocompatible graft further comprises at least one active agent selected from a protein, type I collagen, fibronectin, or TGF-beta 2, heparin, growth factors, antibodies, antimetabolites, chemotherapeutic agents, anti-inflammatory agent, anti-biotic agent, and any combinations thereof.

16. A dental or subperiosteal implant according to claim 1, wherein said synthetic biocompatible graft further comprises at least one non-porous layer.

17. A method of treating at least one of insufficient jawbone, fractured jawbone, edentulous patients with partial absorption of the jawbone, and any similar condition or symptom comprising providing at least one subperiosteal implant comprising at least one synthetic biocompatible graft having a porous polymeric structure with pores of less than 5 microns.

18. (canceled)

19. (canceled)
